(19) United States
(12) Patent Application Publication
(10) Pub. No.: US 2004/0170635 A1
(43) Pub. Date: Sep. 2, 2004

(54) Methods for treating or preventing skin disorders using cd2-binding agents

(76) Inventors: Akshay K. Vaishnaw, Arlington, MA (US); Kevin D. Cooper, Moreland Hills, OH (US); Daniel Schrager, Princeton, NJ (US); Thomas S. Mccormick, Orange Village, OH (US)

Correspondence Address: FISH & RICHARDSON PC, 225 FRANKLIN ST, BOSTON, MA, 02110, US

(21) Appl. No.: 10/470,764

(22) PCT Filed: Jan. 25, 2002
(86) PCT No.: PCT/US02/02314

Related U.S. Application Data
(62) Division of application No. 60/265,964, filed on Feb. 1, 2001.

(51) Int. Cl. A61K 39/395; A61K 31/56; A61K 35/78

(52) U.S. Cl. 424/178.1; 424/725.1; 514/171; 514/183

(57) ABSTRACT

Methods for treating or preventing an epidermal or dermal disorder, e.g., psoriasis, using a CD2-binding agent, e.g., an inhibitor of the CD2/LFA-3 interaction (e.g., an LFA-3/IgG fusion polypeptide), in combination with an auxiliary agent, e.g., UVB irradiation, are disclosed.

### RELATED APPLICATIONS

This application claims priority to U.S. provisional application No. 60/265,964 filed on Feb. 1, 2001, the contents of which are incorporated herein by reference.

### FIELD OF THE INVENTION

The invention relates to the use of a CD2-binding agent, e.g., an inhibitor of the CD2/LFA-3 interaction (e.g., an LFA-3/IgG fusion polypeptide), in combination with an auxiliary agent, e.g., UVB irradiation, to treat a disorder, e.g., psoriasis, or other epidermal or dermal disorders characterized by aberrant T cell activity or proliferation.

### BACKGROUND OF THE INVENTION

Skin disorders, such as psoriasis, eczema, mycosis fungoides, actinic keratosis, and lichen planus, are known to affect one to two percent of the U.S. population, with as many as 150,000-260,000 new cases occurring annually (“Research Needs in 11 Major Areas in Dermatology” I. Psoriasis. J. Invest. Dermatol. 73:402-13, 1979). A number of these skin disorders are characterized by increased T cell activation and abnormal antigen presentation in the dermis and epidermis (Cooper, “Immunoregulation in the Skin”, in Cutaneous Lymphoma, Curr. Probl. Dermatol., eds. van Vloten et al., 19, pp. 69-80 at pp. 73, 74, 76 (1990)). For example, in contact allergic dermatitis, activation of intracutaneous T cells is observed. It is known that skin from patients exhibiting atopic dermatitis contains an increased number of Langerhans' cells (Cooper, “Immunoregulation in the Skin”, in Cutaneous Lymphoma, Curr. Probl. Dermatol., eds. van Vloten et al., 19, at p. 74 (1990)). In psoriatic skin, there is an increased number of antigen presenting cells, composed of both Langerhans' cells and non-Langerhans' cell Class II MHC-bearing antigen presenting cells (Cooper, “Immunoregulation in the Skin”, in Cutaneous Lymphoma, Curr. Probl. Dermatol., eds. van Vloten et al., 19, at p. 75 (1990)).

Cutaneous T cell lymphoma is characterized by the expansion of a malignant clonal population of T cells in the dermis and epidermis. Lesional epidermal cells contain increased numbers of CD1⁺DR⁺ antigen presenting cells (Cooper, “Immunoregulation in the Skin” in Cutaneous Lymphoma, Curr. Probl. Dermatol., eds. van Vloten et al., 19, at pp. 76-77 (1990)).

Presently known therapies for the above mentioned skin diseases are limited. Steroids or cyclosporin A are commonly used in the treatment of psoriasis, lichen planus, urticaria, atopic dermatitis, UV damage, pyoderma gangrenosum, vitiligo, ocular cicatricial pemphigoid, alopecia areata, allergic and irritant contact dermatitis and cutaneous T cell lymphoma. In addition, for some of these skin disorders, various therapies include retinoids, PUVA, nitrogen mustard, interferon, chemotherapy, methotrexate, light therapy (e.g., UV light and PUVA), antibiotics and antihistamines. See generally, Fitzpatrick, Dermatology in General Medicine, 3rd ed., McGraw Hill (1987). UV light therapies, both UVA and UVB therapy, expose the skin to UV radiation between 320-400 nm (UVA radiation) or 290-320 nm (UVB radiation). PUVA therapy is a form of photochemotherapy that involves repeated topical application of psoralen or a psoralen-based compound to an affected region of skin, followed by exposure of that region to UVA radiation. Another method used to treat proliferative skin diseases, particularly psoriasis and mycosis fungoides, is photodynamic therapy (PDT).

Side effects to these therapies are known. Most commonly encountered drawbacks for cyclosporin A include toxicity due to immunosuppression, as well as renal and neural toxicity. Steroids have well known side effects including induction of Cushing Syndrome. Side effects of some of the other aforementioned therapies include skin cancer, bone marrow and constitutional toxicities, ligament calcification, liver fibrosis and other disorders. With respect to light therapy, prolonged treatment of skin diseases using these types of therapies can result in significant acute and chronic adverse effects including erythema, pruritus, skin cancer, and chronic light-induced damage of the skin (Stern et al., N. E. J. of Med. 300:809-812, 1979).

Accordingly, there exists a need for improved therapeutic modalities for preventing and treating skin disorders exhibiting increased T cell activation and abnormal antigen presentation.

### SUMMARY OF THE INVENTION

The invention is based, in part, on the discovery that the combination of a CD2-binding agent, e.g., LFA-3/IgG fusion polypeptide, and an auxiliary agent e.g., UVB irradiation, is highly effective in treating psoriatic lesions. The auxiliary agent is an agent having one or more of the following properties: (i) it reduces interferon-γ (IFN γ) production and/or levels; (ii) it reduces the number of T cells in the affected tissue, particularly CD69⁺ T cells; (iii) it decreases CD40 ligand (CD40L, i.e., CD154) expression; or (iv) it increases T cell death, e.g., apoptosis. While not wishing to be bound by theory, it is believed that the treatment acts by reducing the number and activity of Th1-type cells in psoriatic lesions. Accordingly, the invention provides methods and compositions for treating or preventing epidermal or dermal disorders characterized by aberrant T cell activity or proliferation.

In general, the invention features a method of treating, or preventing, in a subject, a skin disorder, e.g., an epidermal or dermal disorder characterized by aberrant (e.g., increased) T cell, e.g., Th1-type cell, activity or proliferation. The method includes:

Administering to the subject a CD2-binding agent, an LFA-3-binding agent, or an inhibitor of the CD2/LFA-3 interaction, e.g., a CD2-binding agent, in combination with an auxiliary agent, e.g., an agent having one or more of the following properties: (i) it reduces interferon-γ (IFN γ) production and/or levels; (ii) it reduces the number of T cells e.g., memory effector T lymphocytes (e.g., CD8/CD45 RO+ cells or CD4/CD45 RO+ cells) in the affected tissue, particularly CD69⁺ T cells; (iii) it decreases CD40 ligand (CD40L) expression; or (iv) it increases T cell death, e.g., apoptosis, to thereby treat or prevent said skin disorder.

In a preferred embodiment, the skin disorder is characterized by one or more of the following: (i) increased levels of IFN γ, e.g., increased T cell IFN γ production; (ii) elevated levels of T cell populations, e.g., CD3-, CD4-, CD8, CD45-and/or CD69-positive T cells; (iii) increased CD40 ligand expression; or (iii) keratinocyte hyperproliferation.

In a preferred embodiment, the skin disorder is a chronic inflammatory disorder, e.g., psoriasis.

In a preferred embodiment, the skin disorder is an autoimmune disorder, e.g., a chronic autoimmune disorder, e.g., psoriasis.

In a preferred embodiment, the skin disorder is chosen from one or more of: psoriasis, atopic dermatitis, cutaneous T cell lymphoma such as mycosis fungoides, allergic and irritant contact dermatitis, lichen planus, alopecia, e.g., alopecia areata, pyoderma gangrenosum, vitiligo, ocular cicatricial pemphigoid, or urticaria. Preferably, the skin disorder is psoriasis, atopic dermatitis, allergic dermatitis, or alopecia areata. Most preferably, the disorder is psoriasis.

In a preferred embodiment, the CD2-binding agent is an inhibitor of the CD2/LFA-3 interaction, e.g., an anti-CD2 antibody homolog; a soluble CD2-binding fragment of LFA-3; a CD2-binding fragment of LFA-3 coupled, e.g., fused, to another moiety, e.g., all or part of a plasma protein, such as all or part of an immunoglobulin (e.g., an IgG (e.g., an IgG1, IgG2, IgG3, IgG4), IgM, IgA (e.g., IgA1, IgA2), IgD, and IgE, but preferably an IgG) or a fragment thereof (e.g., an immunoglobulin constant region), serum albumin (e.g., human serum albumin), or a synthetic hydrophilic polymer such as pegylation (e.g., PEG); a CD2-binding small molecule or peptidomimetic; a CD2-binding polypeptide fragment identified, e.g., by phage display or using a peptide combinatorial library.

In a preferred embodiment, the CD2 binding agent is a CD2-binding fragment of LFA-3 fused to all or part of an immunoglobulin hinge and heavy chain constant region or a portion thereof (e.g., an LFA-3/IgG fusion polypeptide, e.g., an LFA-3/IgG fusion polypeptide having the nucleotide and amino acid sequence shown in SEQ ID NO:7 and 8 of U.S. Pat. No. 6,162,432, which is hereby incorporated by reference). Yet another preferred LFA-3/IgG fusion protein has the amino acid sequence shown FIG. 1 and is encoded by the nucleotide sequence shown in the same Figure.

In a preferred embodiment, the CD2 binding agent is a soluble LFA-3 polypeptide, e.g., a polypeptides chosen from amino acids 1-92, 1-80, 50-65, 20-80 of the LFA-3 sequence shown in SEQ ID NO: 3 of U.S. Pat. No. 6,162,432, which is hereby incorporated by reference.

In a preferred embodiment, the CD2 binding agent is an anti-CD2 antibody homolog, e.g., a monoclonal anti-CD2 antibody (e.g., a recombinant (e.g. a chimeric or humanized anti-CD2 antibody) or an antigen binding fragment thereof (e.g., a Fab fragment, a Fab′ fragment, a F(ab′) 2 fragment, a F(v) fragment or an intact immunoglobulin heavy chain of an anti-CD2 antibody homolog).

In a preferred embodiment, the CD2 binding agent includes a first moiety that binds CD2 and a second moiety that recruits an effector cell. The first moiety can be a CD2 binding fragment of LFA-3, e.g., a fragment described herein, or an antibody homolog that binds CD2, e.g., an antibody homolog described herein. The second moiety can be a polypeptide capable of recruiting effector cells, such as natural killer (NK) cells. Preferably, the second moiety includes: A fragment of an immunoglobulin constant region, e.g., an immunoglobulin fragment described herein; or an Fc receptor (e.g., FcγRI or FcγRII) binding antibody homolog.

In a particularly preferred embodiment, the CD2 binding agent is a chimeric, e.g., fusion, polypeptide which includes a CD2-binding fragment of LFA-3 and a polypeptide capable of recruiting effector cells. In a preferred embodiment the chimeric or fusion polypeptide includes a CD2 binding fragment of LFA-3, e.g., a fragment described herein, or an antibody homolog which binds CD2, e.g., an antibody homolog described herein, and a fragment of an immunoglobulin constant region, e.g., an immunoglobulin fragment described herein; or an Fc receptor binding antibody homolog.

In a preferred embodiment, the inhibitor of the CD2/LFA-3 interaction is an LFA-3-binding agent, e.g., an anti-LFA-3 antibody homolog; a soluble LFA-3-binding fragment of CD2; an LFA-3-binding fragment of CD2 fused to another moiety, e.g., a plasma protein, such as an immunoglobulin (e.g., an IgG (e.g., an IgG1, IgG2, IgG3, IgG4), IgM, IgA (e.g., IgA1, IgA2), IgD, and IgE, preferably an IgG) or a fragment thereof (e.g., an immunoglobulin constant region), serum albumin (e.g., human serum albumin), or pegylation; an LFA-3-binding small molecule or peptidomimetic; an LFA-3-binding polypeptide fragment identified, e.g., by phage display or using a peptide combinatorial library.

In a preferred embodiment, the LFA-3-binding agent is an anti-LFA-3 antibody homolog, e.g., a monoclonal anti-LFA-3 antibody (e.g., a recombinant (e.g. a chimeric or humanized anti-LFA-3 antibody) or an antigen binding fragment thereof (e.g., a Fab fragment, a Fab′ fragment, a F(ab′) 2 fragment, a F(v) fragment or an intact immunoglobulin heavy chain of an anti-LFA-3 antibody homolog).

In a preferred embodiment, the auxiliary agent is an agent that directly or indirectly causes one or more of: (i) a reduction in interferon-γ (IFN γ) production and/or levels; (ii) a reduction in the number of T cells, e.g., memory effector T lymphocytes (e.g., CD8/CD45 RO+ cells or CD4/CD45 RO+ cells), in the affected tissue, particularly CD69⁺ T cells; (iii) a decrease in CD40 ligand (CD40L) expression; or (iv) an increase in T cell death, e.g., apoptosis. Such effects may result from one or more of: a reduction in the number or activity of T cells, e.g., memory effector T lymphocytes (e.g., CD8/CD45 RO+ cells or CD4/CD45 RO+ cells); a reduction in the number or activity of IFN γ-producing immune cells (e.g., T cells); sequestration or other inactivation of IFN γ; interference with the synthesis and/or secretion of IFN γ by an immune cell; induction of cell-(e.g., effector cell-) mediated killing of a T cell (e.g., an IFN γ-producing immune cell). Exemplary auxiliary agents include: light therapy (e.g., UVA, UVB or PUVA); methotrexate; retinoids; cyclosporine; etretinate; a cytokine inhibitor, e.g., a macrolactam (e.g., pimecrolimus); an IFN γ-binding agent, e.g., an anti-IFN γ antibody or an antigen-binding fragment thereof; an antagonist of IFN γ, or an IFN γ-receptor, e.g., an antibody or antigen-binding fragment thereof which inhibits the IFN γ-receptor interaction, a small molecule or a peptidomimetic inhibitor.

In a preferred embodiment, the auxiliary agent is chosen from: ultraviolet radiation, e.g., UVA or UVB radiation, PUVA radiation, methotrexate, retinoids, cyclosporine, etretinate, a macrolide, a macrolactam (e.g., tacrolimus (FK506) or ascomycin macrolactam (e.g., pimecrolimus), or any combination thereof. A preferred agent is UVB radiation.

In a preferred embodiment, the method further includes the step of monitoring the subject, e.g., for symptoms, or for changes in cytokine levels, e.g., IFNγ, or in an immune cell population (e.g., T cells, e.g., memory effector T lymphocytes (e.g., CD8/CD45 RO+ cells or CD4/CD45 RO+ cells); IFNγ-producing T cells). The subject can be monitored prior to beginning of treatment, during the treatment, or after one or more elements of the treatment have been administered. Monitoring can be used to evaluate the need for further treatment with the same agents or for additional treatment with additional agents. Generally, a decrease in cytokine levels, e.g., IFNγ, or in the selected immune cell population (e.g., T cells, e.g., memory effector T lymphocytes (e.g., CD8/CD45 RO+ cells or CD4/CD45 RO+ cells); or IFNγ-producing T cells) is indicative of the improved disorder of the subject.

Treatment can include combination with yet other agents. Thus, in a preferred embodiment, the method further includes: administering to the subject an agent which inhibits a T cell receptor co-stimulatory signal, e.g., an inhibitor of B7-CD28, ICAM-LFA-1, or CD40-CD40L interaction, or any combination thereof. The agent can be any molecule (e.g., antibody or fragment thereof, soluble polypeptide, small molecule or peptidomimetic that interferes with the ligand (e.g., B7, ICAM, CD40), counterligand (e.g., CD28, LFA-1, CD40L) interaction. Preferably, the agent is an antibody homolog against LFA-1 or CD40L, e.g., a humanized anti-LFA-1 antibody.

In a preferred embodiment, the method further includes: administering to the subject a topically applied agent, e.g., one or more of a steroid, vitamin (e.g., vitamin D), tar, an anthralin, a macrolide, or a macrolactam, e.g., tacrolimus (FK506) or ascomycin macrolactam (e.g., pimecrolimus).

In a preferred embodiment, the subject is a mammal, e.g., a primate, preferably a higher primate, e.g., a human. In one embodiment, the subject is a patient having an epidermal or a dermal disorder (e.g., a patient suffering from a mild, moderate or severe form of psoriasis).

In a preferred embodiment, the skin disorder affects an epidermal, dermal or hypodermal tissue. Preferably, the skin disorder affects an epidermal tissue.

In a preferred embodiment, the CD2-binding agent and the auxiliary agent are administered simultaneously. In other embodiments, the CD2-binding agent and the auxiliary agent are administered sequentially, by e.g., administering the CD2-binding agent first followed by the auxiliary agent, or vice versa. The CD2-binding agent and the auxiliary agent can be administered in combination with topical therapy (e.g., steroid, vitamin (e.g., vitamin D) or tars, anthralin, or a macrolactam, e.g., tacrolimus (FK506) or ascomycin macrolactam (e.g., pimecrolimus).

In other embodiments, the CD2-binding agent and the auxiliary agent are administered rotationally. For example, administration of a CD2-binding agent can be followed by a rotation schedule of topical therapies, e.g., a course of steroid therapy, followed by vitamin (e.g., vitamin D3 treatment, then followed by anthralin. Any combination and sequence of topical agents can be used.

In a preferred embodiment, the CD2-binding agent and the auxiliary agent are administered in sufficiently close proximity, e.g., spatially or temporally, such that the desired effect, e.g., the reduction in the IFNγ levels, or the reduction in a symptom, is greater than what would be observed with the auxiliary agent administered without the CD2-binding agent, or the CD2-binding agent administered without the auxiliary agent.

In a preferred embodiment, the CD2-binding agent is administered during a period wherein the IFNγ levels are reduced by the IFNγ reducing agent. For example, the CD2-binding agent can be administered to a subject, a patient having a mild form of psoriasis, simultaneously, before, or after topical therapy with one or more of a steroid, vitamin (e.g., vitamin D), tars, anthralins, macrolides, or macrolactams, e.g., tacrolimus (FK506) or ascomycin macrolactam (e.g., pimecrolimus), or any combination thereof. In subjects, e.g., patients having moderate to severe forms of psoriasis, the CD2-binding agent can be administered simultaneously, before, or after light therapy (e.g., treatment with UVB and/or PUVA). In other embodiments, the CD2-binding agent can be administered simultaneously, before, or after light therapy in combination with one or more of retinoids, methotrexate or cyclosporine. In one embodiment, a moderate to severe psoriatic patient is treated with a CD2-binding agent at any time during a schedule comprising: light therapy and retinoids, followed by methotrexate, followed by cycosporine.

In some embodiments, the CD2-binding agent is administered systemically (e.g., intravenously, intramuscularly, by an infusion device, or subcutaneously). In other embodiment, the CD2-binding agent is administered locally (e.g., topically) to an affected area, e.g., a psoriatic lesion.

In a preferred embodiment, the CD2-binding agent is an LFA-3/IgG fusion polypeptide and is administered systemically. In one embodiment, the LFA-3/IgG fusion polypeptide is administered to a subject once a week during a therapeutic treatment period of twelve weeks.

In a preferred embodiment, the auxiliary agent is administered locally, e.g., by light exposure, e.g., UVA, UVB or PUVA irradiation. In other embodiments, the auxiliary (e.g., methotrexate, oral retinoids, cyclosporin, macrolactam (e.g., tacrolimus or pimecrolimus) is administered systemically.

In a preferred embodiment, the auxiliary agent is UVB, e.g., ultraviolet light in the range of 290-320 nm, more preferably in the form of narrow band UVB at 311 nm.

Any combination of mode of administration of the CD2-binding agent and the auxiliary agent is within the scope of the invention.

The CD2-binding agent and the auxiliary agent can be administered during periods of active disease, or during a period of remission or less active disease. The CD2-binding agent and the auxiliary agent can be administered before treatment, concurrently with treatment, post-treatment, or during remission of the disease.

In another aspect, the invention features a method of treating, or preventing, psoriasis in a subject. The method includes:

Administering to the subject a fusion polypeptide which includes a CD2-binding fragment of LFA-3 fused to a fragment of the constant region of an IgG, in combination with an amount of UVB sufficient to reduce interferon-γ levels in the epidermal of the subject, to thereby treat or prevent said psoriasis. Advantageously, the present combination treatment method can result in a significantly enhanced degree of disease remission (including clearance) and/or a significantly extended period of disease remission or clearance, relative to that achieved by either agent alone.

In a preferred embodiment, the fragment of LFA-3 is fused to all or part of an immunoglobulin hinge and heavy chain constant region, e.g., an LFA-3/IgG fusion polypeptide encoded by a nucleic acid having the nucleotide sequence shown in SEQ ID NO:7, and having the amino acid sequence shown in SEQ ID NO:8, of U.S. Pat. No. 6,162,432, which is hereby incorporated by reference). Yet another preferred LFA-3/IgG fusion protein has the amino acid sequence shown FIG. 1 and is encoded by the nucleotide sequence shown in the same Figure.

In a preferred embodiment, the CD2 binding LFA-3 polypeptide includes amino acids 1-92, 1-80, 50-65, 20-80 of the LFA-3 sequence shown in SEQ ID NO:3 of U.S. Pat. No. 6,162,432, which is hereby incorporated by reference.

In a preferred embodiment, the method further includes the step of monitoring the subject, e.g., for symptoms, or for changes in cytokine levels, e.g., IFNγ, or in an immune cell population (e.g., e.g., T cells, e.g., memory effector T lymphocytes (e.g., CD8/CD45 RO+ cells or CD4/CD45 RO+ cells); or IFNγ-producing T cells). The subject can be monitored prior to beginning of treatment or after one or more elements of the treatment have been administered. Monitoring can be used to evaluate the need for further treatment with the same agents or for additional treatment with additional agents. Generally, a decrease in cytokine levels, e.g., IFNγ, or in the selected immune cell population (e.g., T cells, e.g., memory effector T lymphocytes (e.g., CD8/CD45 RO+ cells or CD4/CD45 RO+ cells); or IFNγ-producing T cells) is indicative of the improved disorder of the subject.

Treatment can include combination with yet other agents. Thus, in a preferred embodiment, the method further includes: administering to the subject an agent which inhibits a T cell receptor co-stimulatory signal, e.g., an inhibitor of B7/CD28, ICAM-LFA-1, or CD40-CD40L (i.e., CD154), or any combination thereof. The agent can be any molecule (e.g., antibody or fragment thereof, soluble polypeptide, small molecule or peptidomimetic that interferes with the ligand (e.g., B7, ICAM, CD40), counterligand (e.g., CD28, LFA-1, CD40L) interaction. Preferably, the agent is an antibody homolog against LFA-1, e.g., a humanized anto-LFA-1 antibody.

In a preferred embodiment, the method further includes: administering to the subject a topically applied agent, e.g., one or more of, a steroid, vitamin (e.g., vitamin D), tar, or anthralin.

In a preferred embodiment, the subject is a mammal, e.g., a primate, preferably a higher primate, e.g., a human, e.g., a patient having an epidermal or dermal disorder (e.g., a patient suffering from a mild, moderate or severe form of psoriasis).

In a preferred embodiment, the fusion polypeptide and the UVB are administered simultaneously. In other embodiments, the CD2-binding agent and the auxiliary agent are administered sequentially, by e.g., administering the fusion protein first followed by UVB treatment, or vice versa. If the UVB is administered first, the fusion protein should be administered while the UVB therapeutic effects, e.g., a reduction in the level of IFN-γ, is still occurring. If the fusion protein is administered first, the UVB should be administered while the fusion protein's therapeutic effect, e.g., a reduction in the level of IFN-γ, is still occurring.

The fusion polypeptide and the UVB can be administered in combination with topical therapy (e.g., steroid, vitamin (e.g., vitamin D) or tars, anthralins, or macrolactams, e.g., tacrolimus (FK506) or ascomycin macrolactam (e.g., pimecrolimus). In other embodiments, fusion polypeptide and the UVB are administered rotationally. In one embodiment, administration of a CD2-binding agent can be followed by a rotation schedule of topical therapies, e.g., a course of steroid therapy, followed by vitamin (e.g., vitamin D3 treatment, then followed by anthralin.

Any combination and sequence of topical and/or systemic agents can be used.

In a preferred embodiment, fusion polypeptide and the UVB are administered in sufficiently close proximity, e.g., spatially or temporally, such that effect, e.g., a decrease in IFNγ-levels, or the reduction in a symptom, is greater than what would be observed if the UVB were administered without the fusion polypeptide or if the fusion polypeptide were administered without the UVB.

In some embodiments, the fusion polypeptide is administered systemically (e.g., intravenously, intramuscularly, by an infusion (e.g., by an infusion device), or subcutaneously). In one embodiment, the fusion polypeptide is administered to a subject once for a therapeutic treatment period of twelve weeks.

In a preferred embodiment, the auxiliary agent is UVB, e.g., ultraviolet light in the range of 290-320 nm, more preferably in the form of narrow band UVB at 311 nm.

In a preferred embodiment, the UVB is administered locally, e.g., by light exposure, e.g., UVB irradiation.

The fusion polypeptide and/or UVB can be administered during periods of active disease, or during a period of remission or less active disease.

In another aspect, the invention features a method of treating, or preventing, in a subject, a disorder, e.g., an inflammatory disorder. The inflammatory disorder can be a chronic inflammatory disorder, e.g., a chronic inflammatory disorder, characterized by aberrant (e.g., increased) T cell, e.g., Th1-type cell, activity or proliferation. The method includes:

Administering to the subject an inhibitor of the CD2/LFA-3 interaction, e.g., an inhibitor as described herein, in combination with an auxiliary agent, e.g., an agent as described herein, to thereby treat or prevent said chronic inflammatory disorder.

In a preferred embodiment, the method further comprises the step of monitoring the changes in cytokine levels, e.g., IFNγ, or in an immune cell population (e.g., CD8/CD45 RO+ cells or CD4/CD45 RO+ cells); or IFNγ-producing T cells), wherein a decrease in cytokine levels, e.g., IFNγ, or in the immune cell population is indicative of an improved disorder of the subject.

In a preferred embodiment, the chronic inflammatory disorder is psoriasis.

In another aspect, the invention features a method of treating, or preventing, in a subject, an autoimmune disorder. The autoimmune disorder can be a chronic autoimmune disorder, characterized by aberrant (e.g., increased) T cell, e.g., Th1-type cell, activity or proliferation. The method includes:

Administering to the subject an inhibitor of the CD2/LFA-3 interaction, e.g., an inhibitor as described herein, in combination with an auxiliary agent, e.g., an agent as described herein, to thereby treat or prevent said autoimmune disorder.

In a preferred embodiment, the method further comprises the step of monitoring changes in cytokine levels, e.g., IFNγ, or in an immune cell population (e.g., CD8/CD45 RO+ cells or CD4/CD45 RO+ cells); or IFNγ-producing T cells), wherein a decrease in cytokine levels, e.g., IFNγ, or in the immune cell population is indicative of an improved disorder of the subject.

In a preferred embodiment, the autoimmune disorder is psoriasis, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis)).

In a preferred embodiment, the autoimmune disorder affects a cell, a tissue, or an organ at or near a bodily surface, e.g., an epidermal, dermal, ocular, buccal, and/or nasopharyngeal mucosa. In other embodiments, the autoimmune disorder affects a cell, a tissue or an organ that can be accessed using a delivery device, e.g., an endoscope or a needle.

In a preferred embodiment, the autoimmune disorder is chosen from psoriasis or dermatitis (including atopic dermatitis and eczematous dermatitis)).

In another aspect, the invention features a method of treating, or preventing, in a subject, psoriasis. The method includes administering to the subject an inhibitor of the CD2/LFA-3 interaction, e.g., a CD2-binding agent, in combination with an agent selected from the group of irradiation (e.g., UVB or PUVA irradiation), methotrexate, a retinoid (e.g., oral retinoid) and cyclosporin, to thereby treat or prevent psoriasis

In a preferred embodiment, the agent is irradiation, e.g., UVB irradiation.

In yet another aspect, the invention features, a method of modulating (e.g., decreasing) the activity or proliferation of a T cell (e.g., memory effector T lymphocytes (e.g., CD8/CD45 RO+ cells or CD4/CD45 RO+ cells); or IFNγ-producing T cells). The method includes:

Contacting said T cell with an inhibitor of the CD2/LFA-3 interaction, e.g., a CD2-binding agent, in combination with an auxiliary agent, e.g., an agent as described herein, in an amount sufficient to modulate, e.g., decrease, the activity or proliferation of the T cell.

The subject method can be used on cell-free conditions (e.g., a reconstituted system), on cells in culture, e.g. in vitro or ex vivo (e.g., cultures comprising T cells). For example, cells can be cultured in vitro in culture medium and an inhibitor and/or an agent, as described herein, can be introduced to the culture medium. In other embodiment, the T cells are removed from the subject prior to the contacting step. The treated cells can then be returned to the subject. Alternatively, the method can be performed on cells present in a subject, e.g., as part of an in vivo (e.g., therapeutic or prophylactic) therapy protocol.

In another aspect, the invention features a composition (e.g., a pharmaceutical composition), which includes an inhibitor of the CD2/LFA-3 interaction, e.g., an inhibitor of the CD2/LFA-3 interaction as described herein, in combination with an auxiliary agent, e.g., an agent as described herein, and a pharmaceutically acceptable carrier.

In another aspect, the invention features a kit, which includes an inhibitor of the CD2/LFA-3 interaction, e.g., an inhibitor of the CD2/LFA-3 interaction as described herein, in combination with an auxiliary agent, e.g., an agent as described herein, or instructions on how to use the combination of such agents.

In a preferred embodiment, the inhibitor of the CD2/LFA-3 interaction is an LFA-3/Ig fusion polypeptide. Preferably, the LFA-3/Ig fusion polypeptide is lyophilized.

Other features and advantages of the instant invention will become more apparent from the following detailed description and claims.

### BRIEF DESCRIPTION OF THE DRAWINGS

FIG. 1 depicts the amino acid and nucleotide sequences of an LFA-3/IgG fusion protein. The signal peptide corresponds to amino acids 1-28 of FIG. 1; the mature LFA-3 region corresponds to amino acids 29-120 of FIG. 1; and the IgG1 region corresponds to amino acids 121-351 of FIG. 1.

### DETAILED DESCRIPTION OF THE INVENTION

In order that the present invention may be more readily understood, certain terms are first defined. Additional definitions are set forth throughout the detailed description.

As used herein, “CD2” means a CD2 polypeptide that interacts with (e.g., binds to) a naturally occurring LFA-3 polypeptide and which has or is homologous (e.g., at least about 85% homology) to an amino acid sequence as shown in SEQ ID NO:5 of U.S. Pat. No. 6,162,432, which is hereby incorporated by reference; or which is encoded by (a) a naturally occurring mammalian CD2 nucleic acid sequence (e.g., SEQ ID NO:5 of U.S. Pat. No. 6,162,432, which is hereby incorporated by reference); (b) a nucleic acid sequence degenerate to a naturally occurring CD2 nucleic acid sequence; (c) a nucleic acid sequence at least 85% homologous to the naturally occurring mammalian CD2 nucleic acid sequence (e.g., SEQ ID NO:5 of U.S. Pat. No. 6,162,432, which is hereby incorporated by reference); or (d) a nucleic acid sequence that hybridizes to one of the foregoing nucleic acid sequences under conditions equivalent to about 20° C. to 27° C. below Tₘ and 1 M sodium chloride, e.g., a nucleic acid sequence that hybridizes to one of the foregoing nucleic acid sequences under stringent conditions, e.g., highly stringent conditions.

As used herein, “LFA-3” means an LFA-3 polypeptide that binds to a naturally occurring CD2 polypeptide and which has or is homologous (e.g., at least about 85% homology) to an amino acid sequence as shown in SEQ ID NO:1 or 3 of U.S. Pat. No. 6,162,432; or which is encoded by (a) a naturally occurring mammalian LFA-3 nucleic acid sequence (e.g., SEQ ID NO:1 or SEQ ID NO:3 of U.S. Pat. No. 6,162,432, which is hereby incorporated by reference); (b) a nucleic acid sequence degenerate to a naturally occurring LFA-3 nucleic acid sequence; (c) a nucleic acid sequence at least 85% homologous to the naturally occurring mammalian LFA-3 nucleic acid sequence (e.g., SEQ ID NO:1 or SEQ ID NO:3 of U.S. Pat. No. 6,162,432, which is hereby incorporated by reference); or (d) a nucleic acid sequence that hybridizes to one of the foregoing nucleic acid sequences under conditions equivalent to about 20° C. to 27° C. below Tₘ and 1 M sodium chloride, e.g., a nucleic acid sequence that hybridizes to one of the foregoing nucleic acid sequences under stringent conditions, e.g., highly stringent conditions.

A “CD2-binding agent” is an agent that interacts with (e.g., binds to) CD2 and preferably modulates (preferably decreases) the CD2/LFA-3 interaction and/or modulates CD2 signaling. Examples of CD2-binding agents include: soluble LFA-3 binding fragments of a naturally occurring CD2 ligand; soluble fusions of LFA-3 or a CD2 binding fragment thereof to another protein or polypeptide, e.g., an immunoglublin or a fragment thereof, an LFA-3/CD2 fusion polypetide; antibodies which bind CD2, e.g., recombinant, monoclonal, chimeric, CDR-grafted, humanized, human, or rodent antibodies; and small molecule or peptidomimetics.

An “LFA-3-binding agent” is an agent which interacts with (e.g., binds to) LFA-3 and preferably modulates (preferably decreases) the CD2/LFA-3 interaction and/or modulates LFA-3 signaling. Examples of LFA-3-binding agents include: soluble CD2 binding fragments of a naturally occurring LFA-3 ligand; soluble fusions of CD2 or an LFA-3 binding fragment thereof to another protein or polypeptide, e.g., an immunoglublin or a fragment thereof, an LFA-3/CD2 fusion polypetide; antibodies which bind LFA-3, e.g., recombinant, monoclonal, chimeric, CDR-grafted, humanized, human, or rodent antibodies; and small molecule or peptidomimetics.

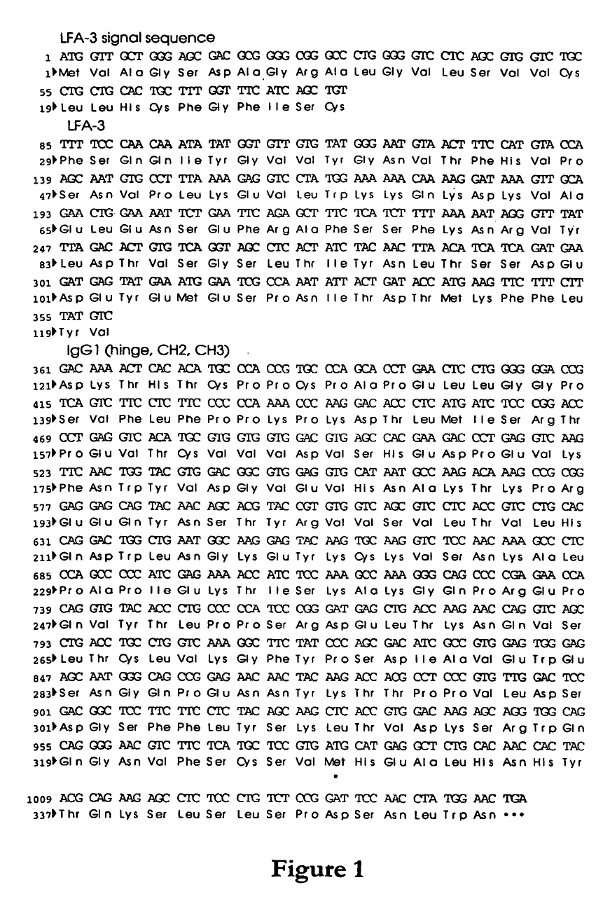

An “LFA-3/IgG” fusion polypeptide is a fusion polypeptide which includes an LFA-3 sequence which binds CD2 and all or a portion of an immunoglobulin sequence, e.g., a portion of an immunoglobulin sequence which interacts with an Fc receptor. The LFA-3 sequence can be full length LFA-3 or a CD2-binding fragment thereof. In a preferred embodiment, the LFA-3 sequence is human LFA-3, and preferably a sequence which is identical to one or both alleles of the subject. Other embodiments can include a modified LFA-3 sequence, e.g., one which differs from a human LFA-3 sequence by at least 1, but less than, 3, 4, 5, or 6 residues. (The complete amino acid sequence of a human LFA-3 is found at SEQ ID NO:1 or 3 of U.S. Pat. No. 6,162,432, which is hereby incorporated by reference). A particularly preferred LFA-3/IgG fusion protein is encoded by a nucleic acid having the nucleotide sequence shown in SEQ ID NO:7, and having the amino acid sequence shown in SEQ ID NO:8, of U.S. Pat. No. 6,162,432, which is hereby incorporated by reference. Yet another preferred LFA-3/IgG fusion protein (also referred to herein as the “large splice product”) has the amino acid sequence shown FIG. 1 and is encoded by the nucleotide sequence shown in the same Figure. The signal peptide corresponds to amino acids 1-28 of FIG. 1; the mature LFA-3 region corresponds to amino acids 29-120 of FIG. 1; and the IgGI region corresponds to amino acids 121 to 351 of FIG. 1.

As used herein, a “soluble LFA-3 polypeptide” or a “soluble CD2 polypeptide” is an LFA-3 or CD2 polypeptide incapable of anchoring itself in a biological membrane. Such soluble polypeptides include, for example, CD2 and LFA-3 polypeptides that lack a sufficient portion of their membrane spanning domain to anchor the polypeptide or are modified such that the membrane spanning domain is non-functional. As used herein soluble LFA-3 polypeptides include full-length or truncated (e.g., with internal deletions) PI-linked LFA-3.

As used herein, an “antibody homolog” is a protein comprising one or more polypeptides selected from immunoglobulin light chains, immunoglobulin heavy chains and antigen-binding fragments thereof which are capable of binding to one or more antigens. The component polypeptides of an antibody homolog composed of more than one polypeptide may optionally be disulfide-bound or otherwise covalently crosslinked. Accordingly, antibody homologs include intact immunoglobulins of types IgA, IgG, IgE, IgD, IgM (as well as subtypes thereof), wherein the light chains of the immunoglobulin may be of types kappa or lambda. Antibody homologs also include portions of intact immunoglobulins that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like.

As used herein, a “humanized recombinant antibody homolog” is an antibody homolog, produced by recombinant DNA technology, in which some or all of the amino acids of a human immunoglobulin light or heavy chain that are required for antigen binding have been substituted for the corresponding amino acids from a nonhuman mammalian immunoglobulin light or heavy chain.

As used herein, a “chimeric recombinant antibody homolog” is an antibody homolog, produced by recombinant DNA technology, in which all or part of the hinge and constant regions of an immunoglobulin light chain, heavy chain, or both, have been substituted for the corresponding regions from another immunoglobulin light chain or heavy chain.

Sequences similar or homologous (e.g., at least about 85% sequence identity) to the sequences disclosed herein are also part of this application. In some embodiments, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

As used herein, the term “homologous” is synonymous with “similarity” and means that a sequence of interest differs from a reference sequence by the presence of one or more amino acid substitutions (although modest amino acid insertions or deletions) may also be present. Presently preferred means of calculating degrees of homology or similarity to a reference sequence are through the use of BLAST and Pfam algorithms available, respectively, through Washington University at http://blast.wustl.edu and http://pfam.wustl.edu, in each case, using the algorithm default or recommended parameters for determining significance of calculated sequence relatedness. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6×sodium chloride/sodium citrate (SSC) at about 45° C., followed by one or more washes in 0.2×SSC, 0.1% SDS at 50° C. Another example of stringent hybridization conditions are hybridization in 6×SSC at about 45° C., followed by one or more washes in 0.2×SSC, 0.1% SDS at 55° C. A further example of stringent hybridization conditions are hybridization in 6×SSC at about 45° C., followed by one or more washes in 0.2×SSC, 0.1% SDS at 60° C. Preferably, stringent hybridization conditions are hybridization in 6×SSC at about 45° C., followed by one or more washes in 0.2×SSC, 0.1% SDS at 65° C. Particularly preferred highly stringent conditions (and the conditions that should be used if the practitioner is uncertain about what conditions should be applied to determine if a molecule is within a hybridization limitation of the invention) are 0.5M sodium phosphate, 7% SDS at 65° C., followed by one or more washes at 0.2×SSC, 1% SDS at 65° C.

It is understood that the polypeptides of the invention may have additional conservative or non-essential amino acid substitutions, which do not have a substantial effect on the polypeptide functions. Whether or not a particular substitution will be tolerated, i.e., will not adversely affect desired biological properties, such as binding activity can be determined as described in Bowie, JU et al. (1990) Science 247:1306-1310.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of a hybrid antibody, without abolishing or more preferably, without substantially altering a biological activity, whereas an “essential” amino acid residue results in such a change.

### Skin Disorders

The methods of this invention are useful to prevent or treat mammalian, e.g., primate, or human, skin disorders characterized by increased T cell activation and abnormal antigen presentation in the dermis and epidermis, by administering inhibitors of the CD2/LFA-3 interaction. Such disorders include psoriasis, UV damage, atopic dermatitis, cutaneous T cell lymphoma such as mycosis fungoides, allergic and irritant coritact dermatitis, lichen planus, alopecia, e.g., alopecia areata, pyoderma gangrenosum, vitiligo, ocular cicatricial pemphigoid, and urticaria. It is to be understood that methods of treatment and prophylaxis of skin disorders such as pyoderma gangrenosum and urticaria are included within the scope of the present invention. These latter skin disorders are also cyclosporin A sensitive dermatoses and therefore involve T cell activation. Preferably, the methods of the invention are used in the prophylaxis or treatment of psoriasis, atopic dermatitis, allergic dermatitis, or alopecia areata, and more preferably, psoriasis.

The methods of the invention may be practiced on any subject, e.g., a mammal, preferably on humans. As used herein, the term “subject” is intended to include human and non-human animals. Preferred human animals include a human patient having a skin disorders characterized by increased T cell activation and abnormal antigen presentation in the dermis and epidermis. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc.

### Inhibitors Of The CD2/LFA-3 Interaction

Any inhibitor of the CD2/LFA-3 interaction is useful in the methods of this invention. Such inhibitors include anti-LFA-3 antibody homologs, anti-CD2 antibody homologs, soluble LFA-3 polypeptides, soluble CD2 polypeptides, small molecules, e.g., (e.g., a chemical agent having a molecular weight of less than 2500 Da, preferably, less than 1500 Da, a chemical, e.g., a small organic molecule, e.g., a product of a combinatorial library), LFA-3 and CD2 mimetic agents and derivatives thereof. Preferred inhibitors are soluble LFA-3 polypeptides and anti-LFA-3 antibody homologs.

The utility in the methods of this invention of specific soluble CD2 polypeptides, soluble LFA-3 polypeptides, anti-LFA-3 antibody homologs, anti-CD2 antibody homologs or CD2 and LFA-3 mimetic agents may easily be determined by assaying their ability to inhibit the LFA-3/CD2 interaction. This ability may be assayed, for example, using a simple cell binding assay that permits visual (under magnification) evaluation of the ability of the putative inhibitor to inhibit the interaction between LFA-3 and CD2 on cells bearing these molecules. Jurkat cells are preferred as the CD2⁺ substrate and sheep red blood cells or human JY cells are preferred as the LFA-3⁺ substrate. The binding characteristics of soluble polypeptides, antibody homologs and mimetic agents useful in this invention may be assayed in several known ways, such as by radiolabeling the antibody homolog, polypeptide or agent (e.g., ³⁵S or ¹²⁵I) and then contacting the labeled polypeptide, mimetic agent or antibody homolog with CD2⁺ of LFA-3⁺ cells, as appropriate. Binding characteristics may also be assayed using an appropriate enzymatically labelled secondary antibody. Rosetting competition assays such as those described by Seed et al. (Proc. Natl. Acad. Sci. USA, 84, pp. 3365-69 (1987)) may also be used.

Anti-LFA-3 and Anti-CD2 Antibody Homologs

Many types of anti-LFA-3 or anti-CD2 antibody homologs are useful in the methods of this invention. These include monoclonal antibodies, recombinant antibodies, chimeric recombinant antibodies, humanized recombinant antibodies, as well as antigen-binding portions of the foregoing.

Among the anti-LFA-3 antibody homologs, it is preferable to use monoclonal anti-LFA-3 antibodies. It is more preferable to use a monoclonal anti-LFA-3 antibody produced by a hybridoma selected from the group of hybridomas having Accession Nos. ATCC HB 10693 (1E6), ATCC HB 10694 (HC-1B11), ATCC HB 10695 (7A6), and ATCC HB 10696 (8B8), or the monoclonal antibody known as TS2/9 (Sanchez-Madrid et al., “Three Distinct Antigens Associated with Human T-Lymphocyte-Mediated Cytolysis: LFA-1, LFA-2 and LFA-3”, Proc. Natl. Acad. Sci. USA, 79, pp. 7489-93 (1982)). Most preferably, the monoclonal anti-LFA-3 antibody is produced by a hybridoma selected from the group of hybridomas having Accession Nos. ATCC HB 10695 (7A6) and ATCC HB 10693 (1E6).

Among the anti-CD2 antibody homologs, it is preferable to use monoclonal anti-CD2 antibodies, such as the anti-CD2 monoclonal antibodies known as the T11₁ epitope antibodies, including TS2/18 (Sanchez-Madrid et al., “Three Distinct Antigens Associated with Human T-Lymphocyte-Mediated Cytolysis: LFA-1, LFA-2 and LFA-3”, Proc. Natl. Acad. Sci. USA, 79, pp. 7489-93 (1982)).

The technology for producing monoclonal antibodies is well known. Briefly, an immortal cell line (typically myeloma cells) is fused to lymphocytes (typically splenocytes) from a mammal immunized with preparation comprising a given antigen, and the culture supernatants of the resulting hybridoma cells are screened for antibodies against the antigen. See generally, Kohler et al., Nature, “Continuous Cultures of Fused Cells Secreting Antibody of Predefined Specificity”, 256, pp. 495-97 (1975). Useful immunogens for the purpose of this invention include CD2-or LFA-3-bearing cells, as well as cell free preparations containing LFA-3, CD2 or counter receptor-binding fragments thereof (e.g., CD2 fragments that bind to LFA-3 or LFA-3 fragments that bind to CD2).

Immunization may be accomplished using standard procedures. The unit dose and immunization regimen depend on the species of mammal immunized, its immune status, the body weight of the mammal, etc. Typically, the immunized mammals are bled and the serum from each blood sample is assayed for particular antibodies using appropriate screening assays. For example, useful anti-LFA-3 or anti-CD2 antibodies may be identified by testing the ability of the immune serum to block sheep red blood cell resetting of Jurkat cells, which results from the presence of LFA-3 and CD2 on the respective surfaces of these cells. The lymphocytes used in the production of hybridoma cells typically are isolated from immunized mammals whose sera have already tested positive for the presence of the desired antibodies using such screening assays.

Typically, the immortal cell line (e.g., a myeloma cell line) is derived from the same mammalian species as the lymphocytes. Preferred immortal cell lines are mouse myeloma cell lines that are sensitive to culture medium containing hypoxanthine, aminopterin and thymidine (“HAT medium”).

Typically, HAT-sensitive mouse myeloma cells are fused to mouse splenocytes using polyethylene glycol (“PEG”) 3350. Hybridoma cells resulting from the fusion are then selected using HAT medium, which kills unfused and unproductively fused myeloma cells (unfused splenocytes die after several days because they are not transformed). Hybridomas producing a desired antibody are detected by screening the hybridoma culture supernatants, for example, for the ability to bind to their respective counter receptor, or for their ability to block Jurkat cell adhesion to sheep red blood cells. Subcloning of the hybridoma cultures by limiting dilution is typically performed to ensure monoclonality.

To produce anti-LFA-3 or anti-CD2 monoclonal antibodies, hybridoma cells that tested positive in such screening assays are cultured in a nutrient medium under conditions and for a time sufficient to allow the hybridoma cells to secrete the monoclonal antibodies into the culture medium. Tissue culture techniques and culture media suitable for hybridoma cells are well known. The conditioned hybridoma culture supernatant may be collected and the desired antibodies optionally further purified by well-known methods.

Alternatively, the desired antibody may be produced by injecting the hybridoma cells into the peritoneal cavity of a pristane-primed mouse. The hybridoma cells proliferate in the peritoneal cavity, secreting the antibody, which accumulates as ascites fluid. The antibody may be harvested by withdrawing the ascites fluid from the peritoneal cavity with a syringe.

Anti-CD2 and anti-LFA-3 antibody homologs useful in the present invention may also be recombinant antibodies produced by host cells transformed with DNA encoding immunoglobulin light and heavy chains of a desired antibody. Recombinant antibodies may be produced by well known genetic engineering techniques. See, e.g., U.S. Pat. No. 4,816,397, which is incorporated herein by reference.

For example, recombinant antibodies may be produced by cloning cDNA or genomic DNA encoding the immunoglobulin light and heavy chains of the desired antibody from a hybridoma cell that produces an antibody homolog useful in this invention. The cDNA or genomic DNA encoding those polypeptides is then inserted into expression vectors so that both genes are operatively linked to their own transcriptional and translational expression control sequences. The expression vector and expression control sequences are chosen to be compatible with the expression host cell used. Typically, both genes are inserted into the same expression vector.

Prokaryotic or eukaryotic host cells may be used. Expression in eukaryotic host cells is preferred because such cells are more likely than prokaryotic cells to assemble and secrete a properly folded and immunologically active antibody. However, any antibody produced that is inactive due to improper folding may be renaturable according to well known methods (Kim and Baldwin, “Specific Intermediates in the Folding Reactions of Small Proteins and the Mechanism of Protein Folding”, Ann. Rev. Biochem., 51, pp. 459-89 (1982)). It is possible that the host cells will produce portions of intact antibodies, such as light chain dimers or heavy chain dimers, which also are antibody homologs according to the present invention.

It will be understood that variations on the above procedure are useful in the present invention. For example, it may be desired to transform a host cell with DNA encoding either the light chain or the heavy chain (but not both) of an antibody homolog. Recombinant DNA technology may also be used to remove some or all of the DNA encoding either or both of the light and heavy chains that is not necessary for CD2 or LFA-3 counter receptor binding. The molecules expressed from such truncated DNA molecules are useful in the methods of this invention. In addition, bifunctional antibodies may be produced in which one heavy and one light chain are anti-CD2 or anti-LFA-3 antibody homologs and the other heavy and light chain are specific for an antigen other than CD2 or LFA-3, or another epitope of CD2 or LFA-3.

Chimeric recombinant anti-LFA-3 or anti-CD2 antibody homologs may be produced by transforming a host cell with a suitable expression vector comprising DNA encoding the desired immunoglobulin light and heavy chains in which all or some of the DNA encoding the hinge and constant regions of the heavy and/or the light chain have been substituted with DNA from the corresponding region of an immunoglobulin light or heavy chain of a different species. When the original recombinant antibody is nonhuman, and the inhibitor is to be administered to a human, substitution of corresponding human sequences is preferred. An exemplary chimeric recombinant antibody has mouse variable regions and human hinge and constant regions. See generally, U.S. Pat. No. 4,816,397; Morrison et al., “Chimeric Human Antibody Molecules: Mouse Antigen-Binding Domains With Human Constant Region Domains”, Proc. Natl. Acad. Sci. USA, 81, pp. 6851-55 (1984); Robinson et al., International Patent Publication PCT/US86/02269; Akira, et al., European Patent Application 184,187; Taniguchi, M., European Patent Application 171,496; Neuberger et al., International Application WO 86/01533; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

Humanized recombinant anti-LFA-3 or anti-CD2 antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an anti-LFA-3 or anti-CD2 antibody. Nucleic acids encoding the humanized antibody, or fragment thereof, can then be cloned into an appropriate expression vector.

Humanized or CDR-grafted antibody molecules or immunoglobulins can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference. All of the CDR's of a particular human antibody may be replaced with at least a portion of a non-human CDR or only some of the CDR's may be replaced with non-human CDR's. It is only necessary to replace the number of CDR's required for binding of the humanized antibody to a predetermined antigen, e.g., LFA-3 or CD2.

Also within the scope of the invention are humanized antibodies, including immunoglobulins, in which specific amino acids have been substituted, deleted or added. In particular, preferred humanized antibodies have amino acid substitutions in the framework region, such as to improve binding to the antigen. For example, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the contents of which are hereby incorporated by reference. Other techniques for humanizing immunoglobulin chains, including antibodies, are described in Padlan et al. EP 519596 A1, published on Dec. 23, 1992.

Human monoclonal antibodies (mAbs) directed against human LFA-3 or CD2 can be generated using transgenic mice carrying the complete human immune system rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al.,1991, Biotechniques 11:152-156). A similar strategy can also been used to amplify human heavy and light chain variable regions from human antibodies (Larrick et al., 1991, Methods: Companion to Methods in Enzymology 2:106-110).

In an illustrative embodiment, RNA is isolated from B lymphocytes, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combination, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

Once displayed on the surface of a display package (e.g., filamentous phage), the antibody library is screened with the antigen, or peptide fragment thereof, to identify and isolate packages that express an antibody having specificity for the antigen. Nucleic acid encoding the selected antibody can be recovered from the display package (e.g., from the phage genome) and subcloned into other expression vectors by standard recombinant DNA techniques.

Specific antibodies with high affinities for a surface protein can be made according to methods known to those in the art, e.g., methods involving screening of libraries (Ladner, R. C., et al., U.S. Pat. No. 5,233,409; Ladner, R. C., et al., U.S. Pat. No. 5,403,484). Further, the methods of these libraries can be used in screens to obtain binding determinants that are mimetics of the structural determinants of antibodies.

In particular, the Fv binding surface of a particular antibody molecule interacts with its target ligand according to principles of protein-protein interactions, hence sequence data for V_{H} and V_{L} (the latter of which may be of the κ or λ chain type) is the basis for protein engineering techniques known to those with skill in the art. Details of the protein surface that comprises the binding determinants can be obtained from antibody sequence information, by a modeling procedure using previously determined three-dimensional structures from other antibodies obtained from NMR studies or crytallographic data. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites, ” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol. 51, op. cit., pp 1-15.

An antigen binding region can also be obtained by screening various types of combinatorial libraries with a desired binding activity, and to identify the active species, by methods that have been described.

In one embodiment, a variegated peptide library is expressed by a population of display packages to form a peptide display library. Ideally, the display package comprises a system that allows the sampling of very large variegated peptide display libraries, rapid sorting after each affinity separation round, and easy isolation of the peptide-encoding gene from purified display packages. Peptide display libraries can be in, e.g., prokaryotic organisms and viruses, which can be amplified quickly, are relatively easy to manipulate, and which allows the creation of large number of clones. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages. Phage display libraries are described above.

Other techniques include affinity chromatography with an appropriate “receptor” to isolate binding agents, followed by identification of the isolated binding agents or ligands by conventional techniques (e.g., mass spectrometry and NMR). Preferably, the soluble receptor is conjugated to a label (e.g., fluorophores, colorimetric enzymes, radioisotopes, or luminescent compounds) that can be detected to indicate ligand binding. Alternatively, immobilized compounds can be selectively released and allowed to diffuse through a membrane to interact with a receptor.

Combinatorial libraries of compounds can also be synthesized with “tags” to encode the identity of each member of the library (see, e.g., W. C. Still et al., International Application WO 94/08051). In general, this method features the use of inert but readily detectable tags, that are attached to the solid support or to the compounds. When an active compound is detected, the identity of the compound is determined by identification of the unique accompanying tag. This tagging method permits the synthesis of large libraries of compounds which can be identified at very low levels among to total set of all compounds in the library.

Anti-CD2 and anti-LFA-3 antibody homologs that are not intact antibodies are also useful in this invention. Such homologs may be derived from any of the antibody homologs described above. For example, antigen-binding fragments, as well as full-length monomeric, dimeric or trimeric polypeptides derived from the above-described antibodies are themselves useful. Useful antibody homologs of this type include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Anti-LFA-3 heavy chains are preferred anti-LFA-3 antibody fragments.

Antibody fragments may also be produced by chemical methods, e.g., by cleaving an intact antibody with a protease, such as pepsin or papain, and optionally treating the cleaved product with a reducing agent. Alternatively, useful fragments may be produced by using host cells transformed with truncated heavy and/or light chain genes. Heavy and light chain monomers may be produced by treating an intact antibody with a reducing agent, such as dithiothreitol, followed by purification to separate the chains. Heavy and light chain monomers may also be produced by host cells transformed with DNA encoding either the desired heavy chain or light chain, but not both. See, e.g., Ward et al., “Binding Activities of a Repertoire of Single Immunoglobulin Variable Domains Secreted from Escherichia coli”, Nature, 341, pp. 544-46 (1989); Sastry et al., “Cloning of the Immunological Repertoire in Escherichia coli for Generation of Monoclonal Catalytic Antibodies: Construction of a Heavy Chain Variable Region-Specific cDNA Library”, Proc. Natl. Acad. Sci. USA, 86, pp. 5728-32 (1989).

Soluble CD2 and LFA-3 Polypeptides

Soluble LFA-3 polypeptides or soluble CD2 polypeptides that inhibit the interaction of LFA-3 and CD2 are useful in the methods of the present invention. Soluble LFA-3 polypeptides are preferred.

Soluble LFA-3 polypeptides may be derived from the transmembrane form of LFA-3, particularly the extracellular domain (e.g., AA₁-AA₁₈₇ of SEQ ID NO:2 of U.S. Pat. No. 6,162,432, which is hereby incorporated by reference). Such polypeptides are described in U.S. Pat. No. 4,956,281 and co-pending U.S. patent application Ser. No. 07/667,971 (which shares a common assignee with the present application), which are herein incorporated by reference. Preferred soluble LFA-3 polypeptides include polypeptides consisting of AA₁-AA₉₂ of SEQ ID NO:2, AA₁-AA₈₀ of SEQ ID NO:2, AA₅₀-AA₆₅ of SEQ ID NO:2 and AA₂₀-AA₈₀ of SEQ ID NO:2, wherein SEQ ID NO:2 is shown in U.S. Pat. No. 6,162,432, which is hereby incorporated by reference. A vector comprising a DNA sequence encoding SEQ ID NO:2 (i.e., SEQ ID NO:1) is deposited with the American Type Culture Collection, Rockville, Md. under Accession No. 75107, wherein of SEQ ID NO:1 and 2 are shown in U.S. Pat. No. 6,162,432, which are hereby incorporated by reference.

The most preferred fusion proteins of this type contain the amino terminal 92 amino acids of mature LFA-3, the C-terminal 10 amino acids of a human IgG1 hinge region containing the two cysteine residues thought to participate in interchain disulfide bonding, and the C_{H}2 and C_{H}3 regions of a human IgG₁ heavy chain constant domain (e.g., SEQ ID NO:8). This fusion protein is referred to herein as “LFA3TIP.” A plasmid, pSAB152, encoding an exemplary LFA3TIP is deposited with American Type Culture Collection, Rockville, Md., under the accession number ATCC 68720. The DNA sequence of the pSAB152 insert is SEQ ID NO:7. SEQ ID NO:7 and 8 are shown in U.S. Pat. No. 6,162,432, which are hereby incorporated by reference.

The amino acid and nucleotide sequences of a longer splice variant of LFA-3TIP than the one shown in U.S. Pat. No. 6,162,432 is depicted in FIG. 1. The signal peptide of the longer LFA-3TIP variant corresponds to amino acids 1-28 of FIG. 1; the mature LFA-3 region corresponds to amino acids 29-120 of FIG. 1; and the IgG1 region corresponds to amino acids 121-351 of FIG. 1. The longer splice variant of LFA-3TIP differs from the shorter variant by having six amino acids added to the C-terminal end.

One way of producing LFA3TIP for use in the methods of this invention is described in co-pending, commonly assigned U.S. patent application Ser. No. 07/770,967. Generally, conditioned culture medium of COS7 or CHO cells transfected with pSAB152 was concentrated using an AMICON S1Y30 spiral cartridge system (AMICON, Danvers, Mass.) and subjected to Protein A-Sepharose 4B (Sigma, St. Louis, Mo.) chromatography. The bound proteins were eluted and subjected to Superose-12 (Pharmacia/LKB, Piscataway, N.J.) gel filtration chromatography.

Superose-12 fractions containing LFA3TIP with the least amount of contaminating proteins, as determined on SDS-PAGE gels and by Western blot analysis, (see, e.g., Towbin et al., Proc. Natl. Acad. Sci. USA, 74, pp. 4350-54 (1979); Antibodies: A Laboratory Manual, pp. 474-510 (Cold Spring Harbor Laboratory (1988)), were pooled and concentrated in a YM30 Centricon (AMICON). LFA3TIP was detected on Western blots using a rabbit anti-LFA-3 polyclonal antiserum, followed by detectably labeled goat anti-rabbit IgG. The purified LFA3TIP of COS7 or CHO cells was a dimer of two monomeric LFA-3-Ig fusion proteins, connected by disulfide bonds.

Another preferred fusion protein consists of the first and second LFA-3 domain fused to the hinge C_{H}2 and C_{H}3 regions of human IgG1, herein referred to as LLFA3-Ig.

Soluble LFA-3 polypeptides may also be derived from the PI-linked form of LFA-3, such as those described in PCT patent application Ser. No. WO 90/02181. A vector comprising a DNA sequence encoding PI-linked LFA-3 (i.e., SEQ ID NO:3) is deposited with the American Type Culture Collection, Rockville, Md. under Accession No. 68788. It is to be understood that the PI-linked form of LFA-3 and the transmembrane form of LFA-3 have identical amino acid sequences through the entire extracellular domain. Accordingly, the preferred PI-linked LFA-3 polypeptides are the same as for the transmembrane form of LFA-3.

Soluble CD2 polypeptides may be derived from full length CD2, particularly the extracellular domain (e.g., AA₁-AA₁₈₅ of SEQ ID NO:6). Such polypeptides may comprise all or part of the extracellular domain of CD2. Exemplary soluble CD2 polypeptides are described in PCT WO 90/08187, which is herein incorporated by reference.

The production of the soluble polypeptides useful in this invention may be achieved by a variety of methods known in the art. For example, the polypeptides may be derived from intact transmembrane LFA-3 or CD2 molecules or an intact PI-linked LFA-3 molecule by proteolysis using specific endopeptidases in combination with exopeptidases, Edman degradation, or both. The intact LFA-3 molecule or the intact CD2 molecule, in turn, may be purified from its natural source using conventional methods. Alternatively, the intact LFA-3 or CD2 may be produced by known recombinant DNA techniques using cDNAs (see, e.g., U.S. Pat. No. 4,956,281 to Wallner et al.; Aruffo and Seed, Proc. Natl. Acad. Sci., 84, pp. 2941-45 (1987); Sayre et al., Proc. Natl. Acad. Sci. USA, 84, pp. 2941-45 (1987)).

Preferably, the soluble polypeptides useful in the present invention are produced directly, thus eliminating the need for an entire LFA-3 molecule or an entire CD2 molecule as a starting material. This may be achieved by conventional chemical synthesis techniques or by well-known recombinant DNA techniques wherein only those DNA sequences which encode the desired peptides are expressed in transformed hosts. For example, a gene which encodes the desired soluble LFA-3 polypeptide or soluble CD2 polypeptide may be synthesized by chemical means using an oligonucleotide synthesizer. Such oligonucleotides are designed based on the amino acid sequence of the desired soluble LFA-3 polypeptide or soluble CD2 polypeptide. Specific DNA sequences coding for the desired peptide also can be derived from the full length DNA sequence by isolation of specific restriction endonuclease fragments or by PCR synthesis of the specified region.

Standard methods may be applied to synthesize a gene encoding a soluble LFA-3 polypeptide or a soluble CD2 polypeptide that is useful in this invention. For example, the complete amino acid sequence may be used to construct a back-translated gene. A DNA oligomer containing a nucleotide sequence coding for a soluble LFA-3 polypeptide or a soluble CD2 polypeptide useful in this invention may be synthesized in a single step. Alternatively, several smaller oligonucleotides coding for portions of the desired polypeptide may be synthesized and then ligated. Preferably, a soluble LFA-3 polypeptide or a soluble CD2 polypeptide useful in this invention will be synthesized as several separate oligonucleotides which are subsequently linked together. The individual oligonucleotides typically contain 5′ or 3′ overhangs for complementary assembly.

Once assembled, preferred genes will be characterized by sequences that are recognized by restriction endonucleases (including unique restriction sites for direct assembly into a cloning or an expression vector), preferred codons taking into consideration the host expression system to be used, and a sequence which, when transcribed, produces a stable, efficiently translated mRNA. Proper assembly may be confirmed by nucleotide sequencing, restriction mapping, and expression of a biologically active polypeptide in a suitable host.

It will be appreciated by those of skill in the art that, due to the degeneracy of the genetic code, DNA molecules comprising many other nucleotide sequences will also be capable of encoding the soluble LFA-3 and CD2 polypeptides encoded by the specific DNA sequences described above. These degenerate sequences also code for polypeptides that are useful in this invention.

The DNA sequences may be expressed in unicellular hosts. As is well known in the art, in order to obtain high expression levels of a transfected gene in a host, the gene must be operatively linked to transcriptional and translational expression control sequences that are functional in the chosen expression host. Preferably, the expression control sequences, and the gene of interest, will be contained in an expression vector that further comprises a bacterial selection marker and origin of replication. If the expression host is a eukaryotic cell, the expression vector should further comprise an additional expression marker useful in the expression host.

The DNA sequences encoding the desired soluble polypeptides may or may not encode a signal sequence. If the expression host is prokaryotic, it generally is preferred that the DNA sequence not encode a signal sequence. If the expression host is eukaryotic, it generally is preferred that a signal sequence be encoded.

An amino terminal methionine may or may not be present on the expressed product. If the terminal methionine is not cleaved by the expression host, it may, if desired, be chemically removed by standard techniques.

A wide variety of expression host/vector combinations may be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including col E1, pCR1, pBR322, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof. Useful vectors for insect cells include pVL 941.

In addition, any of a wide variety of expression control sequences may be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, the control regions of fd coat protein, the promoter for 3-phosphoglycerate kinase or other glycolytic enzymes, the promoters of acid phosphatase, e.g., Pho5, the promoters of the yeast α-mating system and other sequences known to control the expression of genes of prokaryotic or eukaryotic cells or their viruses, and various combinations thereof.

A wide variety of unicellular host cells are useful. These hosts may include well known eukaryotic and prokaryotic hosts, such as strains of E. coli, Pseudomonas, Bacillus, Streptomyces, fungi, yeast, insect cells such as Spodoptera frugiperda (SF9), animal cells such as CHO and mouse cells, African green monkey cells such as COS 1, COS 7, BSC 1, BSC 40, and BMT 10, and human cells, as well as plant cells in tissue culture. For animal cell expression, we prefer CHO cells and COS 7 cells.

It should, of course, be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered.

In selecting an expression control sequence, a variety of factors should also be considered. These include, for example, the relative strength of the sequence, its controllability, and its compatibility with the DNA sequences discussed herein, particularly as regards potential secondary structures. Unicellular hosts should be selected by consideration of their compatibility with the chosen vector, the toxicity of the product coded for by the DNA sequences, their secretion characteristics, their ability to fold the soluble polypeptides correctly, their fermentation or culture requirements, and the ease of purification of the products coded for by the DNA sequences.

Within these parameters, one of skill in the art may select various vector/expression control sequence/host combinations that will express the desired DNA sequences on fermentation or in large scale animal culture, for example with CHO cells or COS 7 cells.

The soluble LFA-3 and CD2 polypeptides may be isolated from the fermentation or cell culture and purified using any of a variety of conventional methods. One of skill in the art may select the most appropriate isolation and purification techniques.

While recombinant DNA techniques are the preferred method of producing useful soluble CD2 polypeptides or soluble LFA-3 polypeptides having a sequence of more than 20 amino acids, shorter CD2 or LFA-3 polypeptides having less than about 20 amino acids are preferably produced by conventional chemical synthesis techniques. Synthetically produced polypeptides useful in this invention can advantageously be produced in extremely high yields and can be easily purified.

Preferably, such soluble CD2 polypeptides or soluble LFA-3 polypeptides are synthesized by solution phase or solid phase polypeptide synthesis and, optionally, digested with carboxypeptidase (to remove C-terminal amino acids) or degraded by manual Edman degradation (to remove N-terminal amino acids). Proper folding of the polypeptides may be achieved under oxidative conditions which favor disulfide bridge formation as described by Kent, “Chemical Synthesis of Polypeptides and Proteins”, Ann. Rev. Biochem., 57, pp. 957-89 (1988). Polypeptides produced in this way may then be purified by separation techniques widely known in the art, preferably utilizing reverse phase HPLC. The use of solution phase synthesis advantageously allows for the direct addition of certain derivatized amino acids to the growing polypeptide chain, such as the O-sulfate ester of tyrosine. This obviates the need for a subsequent derivatization step to modify any residue of the polypeptides useful in this invention.

LFA-3 and CD2 Mimetic or Small Molecule Agents

Also useful in the methods of this invention are LFA-3 and CD2 mimetic agents. These agents which may be peptides, semi-peptidic compounds or non-peptidic compounds (e.g., small organic molecules), are inhibitors of the CD2/LFA-3 interaction. A preferred CD2 and LFA-3 mimetic agents will inhibit the CD2/LFA-3 interaction at least as well as anti-LFA-3 monoclonal antibody 7A6 or anti-CD2 monoclonal antibody TS2/18 (described supra).

In preferred embodiments, the test agent is a member of a combinatorial library, e.g., a peptide or organic combinatorial library, or a natural product library. In a preferred embodiment, the plurality of test compounds, e.g., library members, includes at least 10, 10², 10³, 10⁴, 10⁵, 10⁶, 10⁷, or 10⁸ compounds. In a preferred embodiment, the plurality of test compounds, e.g., library members, share a structural or functional characteristic.

In one embodiment, the invention provides libraries of LPA-3 and/or CD2 inhibitors. The synthesis of combinatorial libraries is well known in the art and has been reviewed (see, e.g., E. M. Gordon et al., J. Med. Chem. (1994) 37:1385-1401; DeWitt, S. H.; Czarnik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; Ellman, J. A. Acc. Chem. Res. (1996) 29:132; Gordon, E. M.; Gallop, M. A.; Patel, D. V. Acc. Chem. Res. (1996)29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/10092, WO93/09668, WO91/07087, WO93/20242, WO94/08051).

Libraries of compounds of the invention can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis”, 2nd edition, Springer-Verlag, Berlin (1993)). To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy results in a library of peptides, e.g., inhibitors, which can be used to prepare a library of test compounds of the invention. In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

Libraries of compounds can be screened to determine whether any members of the library have a desired activity, and, if so, to identify the active species. Methods of screening combinatorial libraries have been described (see, e.g., Gordon et al., J Med. Chem., supra). Soluble compound libraries can be screened by affinity chromatography with an appropriate receptor to isolate ligands for the receptor, followed by identification of the isolated ligands by conventional techniques (e.g., mass spectrometry, NMR, and the like). Immobilized compounds can be screened by contacting the compounds with a soluble receptor; preferably, the soluble receptor is conjugated to a label (e.g., fluorophores, calorimetric enzymes, radioisotopes, luminescent compounds, and the like) that can be detected to indicate ligand binding. Alternatively, immobilized compounds can be selectively released and allowed to diffuse through a membrane to interact with a receptor. Exemplary assays useful for screening the libraries of the invention are described below.

In one embodiment, compounds of the invention can be screened for the ability to interact with a CD2 or LFA-3 polypeptide by assaying the activity of each compound to bind directly to the polypeptide or to inhibit a CD2/LFA-3 interaction, e.g., by incubating the test compound with a CD2 or LFA-3 polypeptide and a lysate, e.g., a T or APC cell lysate, e.g., in one well of a multiwell plate, such as a standard 96-well microtiter plate. In this embodiment, the activity of each individual compound can be determined. A well or wells having no test compound can be used as a control. After incubation, the activity of each test compound can be determined by assaying each well. Thus, the activities of a plurality of test compounds can be determined in parallel.

In still another embodiment, large numbers of test compounds can be simultaneously tested for binding activity. For example, test compounds can be synthesized on solid resin beads in a “one bead-one compound” synthesis; the compounds can be immobilized on the resin support through a photolabile linker. A plurality of beads (e.g., as many as 100,000 beads or more) can then be combined with yeast cells and sprayed into a plurality of “nano-droplets”, in which each droplet includes a single bead (and, therefore, a single test compound). Exposure of the nano-droplets to UV light then results in cleavage of the compounds from the beads. It will be appreciated that this assay format allows the screening of large libraries of test compounds in a rapid format.

Combinatorial libraries of compounds can be synthesized with “tags” to encode the identity of each member of the library (see, e.g., W. C. Still et al., U.S. Pat. No. 5,565,324 and PCT Publication Nos. WO 94/08051 and WO 95/28640). In general, this method features the use of inert, but readily detectable, tags, that are attached to the solid support or to the compounds. When an active compound is detected (e.g., by one of the techniques described above), the identity of the compound is determined by identification of the unique accompanying tag. This tagging method permits the synthesis of large libraries of compounds which can be identified at very low levels. Such a tagging scheme can be useful, e.g., in the “nano-droplet” screening assay described above, to identify compounds released from the beads.

In preferred embodiments, the libraries of compounds of the invention contain at least 30 compounds, more preferably at least 100 compounds, and still more preferably at least 500 compounds. In preferred embodiments, the libraries of compounds of the invention contain fewer than 10⁹ compounds, more preferably fewer than 10⁸ compounds, and still more preferably fewer than 10⁷ compounds.

Derivatized Inhibitors

Also useful in the methods of this invention are derivatized inhibitors of the CD2/LFA-3 interaction in which, for example, any of the antibody homologs, soluble CD2 and LFA-3 polypeptides, or CD2 and LFA-3 mimetic agents described herein are functionally linked (by chemical coupling, genetic fusion or otherwise) to one or more members independently selected from the group consisting of anti-LFA-3 and anti-CD2 antibody homologs, soluble LFA-3 and CD2 polypeptides, CD2 and LFA-3 mimetic agents, cytotoxic agents and pharmaceutical agents.

One type of derivatized inhibitor is produced by crosslinking two or more inhibitors (of the same type or of different types). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another possibility for cross-linking takes advantage of the PI linkage signal sequence in PI-linked LFA-3, or fragments thereof. Specifically, DNA encoding the PI-linkage signal sequence (e.g., AA₁₆₂-AA₂₁₂ of SEQ ID NO:4) is ligated downstream of DNA encoding a desired polypeptide, preferably a soluble LFA-3 polypeptide. If this construct is expressed in an appropriate eukaryotic cell, the cell will recognize the PI linkage signal sequence and will covalently link PI to the polypeptide. The hydrophobic property of the PI may then be exploited to form micellar aggregates of the polypeptides.

Also useful are inhibitors linked to one or more cytotoxic or pharmaceutical agents. Useful pharmaceutical agents include biologically active peptides, polypeptides and proteins, such as antibody homologs specific for a human polypeptide other than CD2 or LFA-3, or portions thereof. Useful pharmaceutical agents and cytotoxic agents also include cyclosporin A, prednisone, FK506, methotrexate, steroids, retinoids, interferon, and nitrogen mustard.

Preferred inhibitors derivatized with a pharmaceutical agent include recombinantly-produced polypeptides in which a soluble LFA-3 polypeptide, soluble CD2 polypeptide, or a peptidyl CD2 or peptidyl LFA-3 mimetic agent is fused to all or part of an immunoglobulin heavy chain hinge region and all or part of a heavy chain constant region. Preferred polypeptides for preparing such fusion proteins are soluble LFA-3 polypeptides. Most preferred are fusion proteins containing AA₁-AA₉₂ of LFA-3 (e.g., SEQ ID NO:2) fused to a portion of a human IgG₁ hinge region (including the C-terminal ten amino acids of the hinge region containing two cysteine residues thought to participate in interchain disulfide bonding) and the C_{H}2 and C_{H}3 regions of an IgG₁ heavy chain constant domain. Such fusion proteins are expected to exhibit prolonged serum half-lives and enable inhibitor dimerization.

### Combination Therapy

The binding agents, e.g., CD2-or LFA-3 binding agents, may be used in combination with other therapies, such as such as light therapy (e.g., UVA, UVB or PUVA); chemotherapy (e.g., methotrexate; retinoid; cyclosporine; etretinate); or topical therapy (e.g., steroid, vitamin (e.g., vitamin D), tar, anthralin, a macrolide, or a macrolactam (e.g., tacrolimus or pimecrolimus). Such combination therapy may advantageously utilize lower dosages of the therapeutic or prophylactic agents.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. E.g., the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder, e.g., reduction in IFN γ level or production, induction of T cell apoptosis, or decrease in CD40L expression, is greater than what would be observed with one treatment delivered in the absence of the other. The effect of the two treatments can be partially additive, wholly additive, or greater than additive. The delivery can be such that an effect of the first treatment delivered is still detectable when the second is delivered, e.g., when UVB is delivered first, a reduction in IFN γ is still detectable when LFA-3/Ig fusion is delivered.

In a preferred embodiment a delivery of the first treatment and a delivery of the second treatment occur within 1, 2, 5, 10, 15, or 30 days of one another.

The binding agents as described herein can be used as an adjunct to conventional treatments of skin disorders, such as psoriasis. For example, binding agents can be introduced prior to, concurrently with, or after sequential therapy of psoriasis (reviewed in Koo, J. (1999) J Am Acad Dermatol. 41(3 Pt 2):S25-8). The term “sequential therapy” refers to a treatment strategy involving the use of specific therapeutic agents in a deliberate sequence to optimize the therapeutic outcome. The rationale for this strategy in psoriasis is that it is a chronic disease requiring long-term maintenance therapy, as well as quick relief of symptoms and that some therapies available for psoriasis are better suited for rapid clearance while others are more appropriate for long-term maintenance. Sequential therapy involves 3 main steps: (1) the clearing, or “quick-fix” phase; (2) the transitional phase; and (3) the maintenance phase.

One example of sequential systemic therapy involves the use of a rapidly acting auxiliary agent, e.g., cyclosporine at maximum dermatologic dose (5 mg/kg daily), or methotrexate. After about 1 month, the transitional phase is initiated with the gradual introduction of a CD2-binding agent and/or another auxiliary agent, e.g., acitretin, as a maintenance agent. Once the maximum tolerated dose of a CD2-binding agent and/or another auxiliary agent, e.g., acitretin has been established, the rapidly acting auxiliary agent, e.g., cyclosporine, is gradually tapered and the CD2-binding agent and/or another auxiliary agent, e.g., acitretin, is continued for long-term maintenance. A combination with phototherapy (UVB or PUVA) can be added for improved control if needed.

In other exemplary embodiments, a CD2-binding agent can be administered over an extended period of time (e.g., a therapeutic treatment period of twelve weeks). During periods of remission or less active disease, the CD2-binding agent can be administered alone or in combination with a topical agent (e.g., e.g., steroid, vitamin (e.g., vitamin D), tar, anthralin, a macrolide, or a macrolactam (e.g., tacrolimus (FK506) or pimecrolimus)) and/or phototherapy (e.g., UVA, UVB or PUVA, but preferably, UVB). During periods of active disease, a rapidly acting, but toxic auxiliary agent, such as methotrexate and/or cyclosporin, can be administered for a short treatment period.

Ascomycin macrolactam derivatives such as pimecrolimus (ASM 981 cream 1%) are selective inhibitors of inflammatory cytokines that are currently used in treating inflammatory skin disorders, such as atopic dermatitis, allergic contact dermatitis, irritant contact dermatitis and psoriasis (Stuetz, A. et al. (2001) Semin. Cutan. Med. Surg. 20(4):233-41; Bornhovd, E. et al. (2001) J. Am. Dermatol. 45(5):736-43)

In a preferred embodiment, the CD2-binding agent (e.g., LFA-3/Ig fusion) or a pharmaceutical composition containing the same is administered systemically (e.g., intravenously, intramuscularly, subcutaneously, intra-articularly, intrathecally, periostally, intratumorally, intralesionally, perilesionally by infusion (e.g., using an infusion device), orally, topically or by inhalation). Preferably, the CD2-binding agent is administered intramuscularly or intravenously. In other embodiment, the CD2-binding agent is administered locally (e.g., topically) to an affected area, e.g., a psoriatic lesion.

Light Therapy

In one embodiment, the binding agent as disclosed herein, e.g., the CD2-binding agent described herein, is administered in combination with phototherapy (also referred to herein as “light therapy”). Phototherapy utilizes optical absorption of ultraviolet (UV) radiation by the skin to kill rapidly growing cells and arrest proliferation. At present, both UVA and UVB therapy, which expose the skin to UV radiation between 320-400 nm (UVA radiation) or 290-320 nm (UVB radiation), are effectively and widely used to treat skin disorders. In a preferred embodiment, UVB radiation in the range of 290-320 nm, and more preferably in the form of narrow band UVB at 311 nm is used. In other embodiments, PUVA therapy, a form of photochemotherapy that involves repeated topical application of psoralen or a psoralen-based compound to an affected region of skin, followed by exposure of that region to UVA radiation, can also be used. In yet other embodiments, photodynamic therapy (PDT) can be used to treat skin disorders, particularly psoriasis and mycosis fungoides. In this method, a photosensitizing agent, which is a drug selectively retained in carcinoma cells, is administered to a subject. Following absorption of light (typically between 320-700 nm, depending on the drug) the photosensitizing agent undergoes a photochemical reaction, resulting in the production of cytotoxic singlet oxygen which eventually leads to tumor vessel destruction in the skin (Anderson, et al. (1992) Arch. Dermatol. 128:1631-1636).

In many cases there will be repeated delivery of one, or both, the CD2-binding agent, e.g., LFA-3/Ig fusion, and light therapy, e.g., UVB. The CD2-binding agent can be delivered at equal or unequal time intervals. E.g., the CD2-binding agent can be delivered every 3-12 days, e.g., once a week. Delivery can be repeated as many as 3, 6, 12, 15, 24, or more times. One or more courses of the second treatment, e.g., the delivery of light therapy, e.g., UVB, can precede, follow, or be superimposed simultaneously on, the course of fusion protein delivery.

### Pharmaceutical Compositions

This invention provides a method for preventing or treating the above-mentioned skin disorders in a subject by administering to the mammal one or more CD2-binding agents, e.g., inhibitors of the CD2/LFA-3 interaction, or derivatized form(s) thereof, in combination with an auxiliary agent.

Preferably, an effective amount of the CD2-binding agents or derivatized form thereof is administered. By “effective amount” is meant an amount capable of lessening the spread or severity of the skin disorders described herein.

It will be apparent to those of skill in the art that the effective amount of inhibitor will depend, inter alia, upon the administration schedule, the unit dose administered, whether the inhibitor is administered in combination with other therapeutic agents, the immune status and health of the patient, the therapeutic or prophylactic activity of the particular inhibitor administered and the serum half-life.

Preferably, the CD2-binding agents is administered at a dose between about 0.001 and about 50 mg inhibitor per kg body weight, more preferably, between about 0.01 and about 10 mg inhibitor per kg body weight, most preferably between about 0.1 and about 4 mg inhibitor per kg body weight.

Unit doses should be administered until an effect is observed. The effect may be measured by a variety of methods, including, in vitro T cell activity assays and clearing of affected skin areas. Preferably, the unit dose is administered about one to three times per week or one to three times per day. More preferably, it is administered about one to three times per day for between about 3 and 7 days, or about one to three times per day for between about 3 and 7 days on a monthly basis. It will be recognized, however, that lower or higher dosages and other administrations schedules may be employed.

The CD2-binding agent(s) or derivatized form(s) thereof are also preferably administered in a composition including a pharmaceutically acceptable carrier. By “pharmaceutically acceptable carrier” is meant a carrier that does not cause an allergic reaction or other untoward effect in patients to whom it is administered.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the inhibitor.

As described above, the pharmaceutical composition or CD2-binding agent may be administered in conjunction with other auxiliary therapeutic or prophylactic agents. These include, for example, cyclosporin A, steroids, retinoids, nitrogen mustard, interferon, methotrexate, antibiotics and antihistamines.

These auxiliary agents may be administered in single dosage form with the inhibitor (i.e., as part of the same pharmaceutical composition), a multiple dosage form separately from the inhibitor, but concurrently, or a multiple dosage form wherein the two components are administered separately but sequentially. Alternatively, the CD2-binding agent and the other active agent may be in the form of a single conjugated molecule. Conjugation of the two components may be achieved by standard cross-linking techniques well known in the art. A single molecule may also take the form of a recombinant fusion protein. In addition, the inhibitors, or pharmaceutical compositions, useful in the present invention may be used in combination with other therapies such as PUVA, chemotherapy and UV light. Such combination therapies may advantageously utilize lower dosages of the therapeutic or prophylactic agents.

The CD2-binding agent, or pharmaceutical composition, may be in a variety of forms. These include, for example, solid, semi-solid and liquid dosage forms, such as tablets, pills, powders, liquid solutions, dispersions or suspensions, liposomes, suppositories, injectable, infusible, and topical preparations. The preferred form depends on the intended mode of administration and therapeutic application. The preferred forms are injectable or infusible solutions.

The invention includes formulations suitable for use as topically applied sunscreens or UV-protectants. Preferred embodiments include LFA3TIP preparations. The active ingredient can be formulated in a liposome. The product can be applied before, during, or after UV exposure, or before, during, or after the development of redness.

### Kits

In another aspect, the invention provides kits which include a CD2-binding agent as described herein, in combination with an auxiliary agent, e.g., an agent as described herein, or instructions on how do use such an agent.

In a preferred embodiment, the inhibitor of the CD2/LFA-3 interaction is an LFA-3/Ig fusion polypeptide. Preferably, the LFA-3/Ig fusion polypeptide is lyophilized.

The following invention is further illustrated by the following examples, which should not be construed as further limiting. The contents of all references, pending patent applications and published patents, cited throughout this application are hereby expressly incorporated by reference.

### EXAMPLES

### Example 1

### In Vitro Inhibition of IFNγ and Decrease in CD25 Levels Following LFA3TIP Co-culture

In vitro examination of LFA3TIP using peripheral blood mononuclear cells (PBMC's) from non-psoriatic and psoriatic volunteers demonstrated significant inhibition of IFNγ as well as a decrease in CD25 levels following LFA3TIP co-culture. The decrease in IFNγ levels were also reflected in the in vivo examination of T cells from LFA3TIP treated patients. Given the significant effect of LFA3TIP on PMBC's in vitro, we wished to determine whether or not in vivo LFA3TIP administration had an effect on PBMC's from the treated volunteers. In order to address this, a peripheral blood draw was added to the protocol timed to coincide with each keratome sample to allow us to examine the IFNγ and CD25 levels in PBMC's from the LFA3TIP treated population. PBMC preparations were performed over Ficoll and stimulated in accordance with PBMC protocols known in the art. PBMC's obtained from the in vivo LFA3TIP treated patients were used in further flow cytometry experiments. The peripheral blood experiments add an additional 15 experimental points to the project for each patient, at each time point. This will consist of staining for CD3, CD69, CD25, CD2, IFN gamma, CD40L, and Apo2.7. Additionally, CD40L expression was also be examined on PBMC's from the in vivo treated patients.

### Example 2

### Human LFA-3/IgG1 Fusion Protein Inhibits IFNγ Production by Normal and Psoriatic Peripheral Blood T cells and Enhances the Action of UVB.

Psoriasis is mediated, in part, by activated T cell production of interferon gamma (IFNγ). Alefacept (human LFA-3/IgG1 fusion protein, LFA3TIP, currently being developed by Biogen, Inc. under the brandname Amevive™) shows inhibitory effects on T cells in vitro and in vivo. Phase 3 clinical trials of alefacept are ongoing in psoriasis. UVB irradiation remains one of the most effective treatments of psoriasis, and we have previously reported that a single in vivo UVB exposure can selectively decrease T cell IFNγ production.

To investigate effects of alefacept on T cell IFNγ production, PBMC from normal individuals (n=7) or psoriatic patients (n−7) were activated and IFNγ production was measured by flow cytometry. For 8 μg/ml alefacept-treated non-psoriatic PBMC, the number of IFNγ⁺ T cells decreased in 5/7 cases (20˜90% reduction), increased in 1/7 or remained unchanged in 1/7 cases. In psoriatic PBMC, 8 μg/ml alefacept treatment caused a decrease in IFYγ production in 6/7 patients tested, with a mean 56±0.12% reduction, (p<0.005). PBMC populations could be divided into two groups based upon IFNγ production, high (>10%) or low (<10%) IFNγ^{+CD3+}. When considered separately, both non-psoriatic and psoriatic high producers were effectively inhibited by 8 μg/ml alefacept, with a mean reduction of 56% and 65% respectively. By contrast, low producers showed little inhibition. Anti-Fcγ RI and RIII mAb pretreatment abolished the reduction of IFNγ by alefacept. When PBMC populations were pretreated with UVB irradiation (0˜20 ml/cm²), alefacept enhanced UVB-induced apoptosis and further decreased IFNγ by 32.2% (p=0.009, n=3).

These results indicate that alefacept inhibits T cell IFNγ production, that an interaction with FcγR bearing cells is required, and that its combination with UVB may prove effective in reducing the number and activity of Th1-type cells in the psoriatic lesion.

### Example 3

### Human LFA-3/IgG1 Fusion Protein Treatment for Psoriasis Reduces the Number of Infiltrating IFNγ⁺-Producing T Cells in Lesional Skin

Psoriasis is a chronic inflammatory skin disease mediated, in part, through IFNγ production by activated lesional T cells (Th₁ skewed). Alefacept (human LFA-3/IgG1 fusion protein, LFA3TIP, currently being developed under the brand name AMEVIVE™) is a novel recombinant protein, which leads to a reversible reduction in CD3+CD45RO+blood T cells.

To study effects on skin T cells, an open-label alefacept Phase III psoriasis study was conducted on 6 patients given alefacept once weekly 7.5 mg IV, for 12 consecutive weeks. The percentages of CD3⁺ T cells and IFNγ-producing CD3⁺ populations in total epidermal or dermal cells were analyzed by flow cytometry, and CD3⁺ or IFNγ⁺CD3⁺ cell densities as cell number/mm² were calculated.

In the 5/6 patients demonstrating clinical improvement at week 13, the density of epidermal T cells producing IFNγ (IFNγ⁺CD3⁺) was reduced to 0.26±0.3% of baseline. The mean density IFNγ⁺CD3⁺ cells for all 6 patients at baseline was 182±91/mm² versus 77±45/mm2 at 12 weeks of alefacept treatment (p=0.05). For all 6 patients, the PASI improvement correlated with % change in IFNγ⁺CD3⁺ epidermal cells at r=0.80, p=0.06. Interestingly, in the initial phase of treatment, several patients demonstrated transient increases in IFNγ⁺CD3⁺ lesional T cells at week 3, in association with a transient increase in epidermal thickness; both the IFNγ⁺CD3⁺ T cells and epidermal thickness then decreased in subsequent weeks.

Our results suggest that alefacept can significantly reduce the number of infiltrating Th1-type IFNγ⁺CD3⁺ T cells, in association with clinical improvement. Because IFNγ is believed to be a key factor in the pathogenesis of psoriasis, the reduction of Th₁ cells in the skin may represent a critical step in the clinical improvement of psoriasis.

Deposits

Murine hybridoma cells and anti-LFA-3 antibodies useful in the present invention are exemplified by cultures deposited under the Budapest Treaty with American Type Culture Collection, Rockville, Md., U.S.A., on Mar. 5, 1991, and identified as:

| Designation | ATCC Accession No. |
|---|---|
| 1E6 | HB 10693 |
| HC-1B11 | HB 10694 |
| 7A6 | HB 10695 |
| 8B8 | HB 10696 |

A bacteriophage carrying a plasmid encoding transmembrane LFA-3 was deposited under the Budapest Treaty with In Vitro International, Inc., Linthicum, Md., U.S.A., on May 28, 1987 under Accession No. IVI-10133. This deposit was transferred to American Type Culture Collection on Jun. 20, 1991 and identified as:

| Designation | ATCC Accession No. |
|---|---|
| λHT16[λgt10/LFA-3] | 75107 |

E. coli transformed with a plasmid encoding PI-linked LFA-3 was deposited under the Budapest Treaty with In Vitro International, Inc. on Jul. 22, 1988 under Accession No. IVI-10180. This deposit was transferred to American Type Culture Collection on Jun. 20, 1991 and identified as:

| Designation | ATCC Accession No. |
|---|---|
| p24 | 68788 |

Sequences

The following is a summary of the sequences described in U.S. Pat. No. 6,162,432 and referred to throughout the application:

SEQ ID NO:1 DNA sequence of transmembrane LFA-3

SEQ ID NO:2 Amino acid sequence of transmembrane LFA-3

SEQ ID NO:3 DNA sequence of PI-linked LFA-3

SEQ ID NO:4 Amino acid sequence of PI-linked LFA-3

SEQ ID NO:5 DNA sequence of CD2

SEQ ID NO:6 Amino acid sequence of CD2

SEQ ID NO:7 DNA sequence of LFA3TIP

SEQ ID NO:8 Amino acid sequence of LFA3TIP

### Equivalents

Those skilled in the art will recognize, or be able to ascertain using no more than routine experimentation, many equivalents of the specific embodiments of the invention described herein. Such equivalents are intended to be encompassed by the following claims.

Other embodiments are within the following claims.

What is claimed is:

1. A method of treating, or preventing, in a subject, a epidermal or dermal disorder characterized by aberrant T cell activity or proliferation, comprising:
administering to the subject an inhibitor of the CD2/LFA-3 interaction, in combination with narrow band UVB radiation, to thereby treat or prevent said epidermal dr dermal disorder.

2. The method of claim 1, wherein the epidermal or dermal disorder is characterized by increased T cell IFN γ production.

3. The method of claim 1, wherein the epidermal or dermal disorder is a chronic inflammatory disorder.

4. The method of claim 1, wherein the epidermal or dermal disorder is an autoimmune disorder.

5. The method of claim 1, wherein the epidermal or dermal disorder is psoriasis.

6. The method of claim 1, wherein the inhibitor of the CD2/LFA-3 interaction is a CD2-binding agent.

7. The method of claim 1, wherein the inhibitor of the CD2/LFA-3 interaction is a CD2-binding fragment of LFA-3 fused to an immunoglobulin or a fragment thereof.

8. The method of claim 1, wherein the inhibitor of the CD2/LFA-3 interaction is an LFA-3/IgG fusion polypeptide.

9. The method of claim 1, further comprising the step of monitoring the subject for symptoms, or for changes in cytokine levels or in an immune cell population.

10. The method of claim 1, further comprising the step of administering to the subject a topically applied agent selected from the group consisting of a steroid, vitamin, tar, anthralin, and macrolactam.

11. The method of claim 1, wherein the subject is a mammal.

12. A method of treating, or preventing, psoriasis in a subject, comprising:
administering to the subject a fusion polypeptide which includes a CD2-binding fragment of LFA-3 fused to a fragment of the constant region of an IgG, in combination with an amount of narrow band UVB sufficient to reduce interferon-γ levels in the epidermis of the subject, to thereby treat or prevent said psoriasis.

13. A method of treating, or preventing, in a subject, an inflammatory disorder, comprising:
administering to the subject an inhibitor of the CD2/ILFA-3 interaction, in combination with narrow band UVB radiation, to thereby treat or prevent said inflammatory disorder.

14. A method of treating, or preventing, in a subject, an autoimmune disorder, comprising:
administering to the subject an inhibitor of the CD2/LFA-3 interaction, in combination with narrow band UVB radiation, to thereby treat or prevent said autoimmune disorder.

15. The method of claim 14, wherein the autoimmune disorder is selected from the group consisting of psoriasis, diabetes mellitus, arthritis, rheumatoid arthritis, juvenile rheumatoid arthritis, osteoarthritis, psoriatic arthritis, multiple sclerosis, encephalomyelitis, myasthenia gravis, systemic lupus erythematosis, autoimmune thyroiditis, dermatitis, atopic dermatitis and eczematous dermatitis.

16. A method of treating, or preventing, atopic dermatitis in a subject, comprising:
administering to the subject a fusion polypeptide which includes a CD2-binding fragment of LFA-3 fused to a fragment of the constant region of an IgG, in combination with an amount of narrow band UVB sufficient to reduce interferon-γ levels in the epidermis of the subject, to thereby treat or prevent said atopic dermatitis.

17. The method of claim 1, wherein one or both of the inhibitor of the CD2/LFA-3 interaction and the narrow band UVB radiation is administered repeatedly.

18. The method of claim 1, wherein administration of the narrow band UVB radiation precedes administration of the inhibitor of the CD2/LFA-3 interaction.

19. The method of claim 1, wherein administration of the narrow band UVB treatment follows administration of the inhibitor of the CD2/LFA-3 interaction.

20. The method of claim 1, wherein administration of the narrow band UVB treatment is simultaneous with administration of the inhibitor of the CD2/LFA-3 interaction.

21. The method of claim 1, wherein the narrow band UVB treatment and the inhibitor of the CD2/LFA-3 interaction are administered at unequal time intervals.
